SEQUENCE LISTING (2 sequences)

SEQ ID NO: 1 - protein, 881 aa, Lactobacillus mucosae
MKNLVAKPQGNQLNIYNGNTVLKTLGPGTWENMAFAQDSSAINNINGYLSYTGWYRPYGTSQDGKTWYQTTAMDWRPLLMYTWPSKDVQAQFIKYFVNHGYENANYGLTKVSVANLNKDTDATVLNTAAQNLRYVIEQSIATNKGTGKLANDINGFAATVPELSASSELSLQSMPNYKPDESGTVDSDQVIFVNNDSKDQRRGNTSYADSKYRLMNRTINNQTGNDNSDNSPELLVGNDIDNSNPVVQAENLNWEYFLLNYGKLMGYNQDGNFDGFRIDAADNINADVLDQMGQLMNDMYHMKGNPQNANNHLSYNEGYHSGAAQMLNEKGNPQLYMDSGEFYTLENVLGRANNRDNISDLVTNSIVNRQNDVTENEATPNWSFVTNHDQRKNLINRLIIKDHPGIAYIMGSAYKAEYANQAWQEFYADQKKTDKQYAQYNVPAQYAILLSNKDTVPQIYYGDLYNETAQYMQGKSIYYDAITTLMKARKQFVSGGQTMTKLSDNLIASVRYGKGVANANSEGTDSLSRTSGMAVIVGNNPQMAEQTISINMGRAHANEQYRNLLDTTDNGLTYNADGAENPETLTTDDNGILKVTVKGYSNPYVSGYLGVWVPVVSGNQDVTTNAATVSADSNKIFESNAALDSHMIYEDFSLYQPEPTSTENHAYNIIAQNAELFNNLGITDFWMAPAYTPFGMSRYNEGYSMTDRYNLGTNANPTKYGSGEELANAIAALHSAGLKVQEDIVMNQMIGFSGQEAVTVTRTDDRGMQIYVNGKTYANQIYFAYTTGGGNGQETYGGKYLSELQSKYPDLFTTKAISTGVAPDPTTHITKWSAKYENGTSLQNIGIGLAVKLPNGEYAYLRSSDNKSFNTLLPSEISVKL

SEQ ID NO: 2 - protein, 1593 aa, Lactobacillus fermentum
MEFKKHFKLYKSGKQWCIAAVVTFTATLGLVAGNTTVNADTLTTGGSTPQAQVNQEKGITNTSQPASDQGKTSNTGDVRNTVPQPNNSDKTNQYQADTLTTGGSTPQAQVNQEKGITNTSQPASDQGKTSNTGDVRNTVPQPNNSGKTNQSQNGQDQKESAGNDNGSTGNNDQNKGQQTTDWQKNANNQWVYNGKTDQDLKGTQYVQLPTIPDTNVQGNTNWYFVKDGIAQSGVQQWAGTYYYFDPTTYLRVDNDYRQSQWGDWYLFGNDGRILSGVQQWAGTYYYFDPTTYLRVDDDYVTSQWGLKYMFGKDGRIATGLYKWDKNNQWYYFNPTTYLAVTNNYIQANDGHWYLFTADGTAASRVAKWAGTYYYFDPQTHLRVDDNYVQSQWGDWYMFGKDGRIATGLYKWDKNNQWYYFDPTTYLAVTNNYIQANDGHWYLFTADGTAASRVAKWAGTYYYFDPVTHLRVDNNYVQSQWGDWYLFGNDGRILSGVQQWAGTYYYFDPTTYLRVDDDYVTSQWGLKYMFGKDGRIATGLYKWDKNNQWYYFDPTTYLAVTNNYIQANDGHWYLFTADGTAASRVAKWAGTYYYFDPQTHLRVDDNYVQSQWGDWYMFGKDGRIATGLYKWDKNNQWYYFDPVTYLKVTNKWVDGNYYDEDGAQAISKLVTINNRLYYFDDQGKEISNQFRTIHGDKYYFGNDSAAVTGQQTIDGKVYKFSNYGYLLGNRYGKIENGKLNIYSLADHSLIKTVEAGPWENMAYSMDSNSINNIDGYISYTGWYRPYGTSQDGKTWYPTTVADWRPILMYVWPSKDVQVKFIQYFVNHGYENSNYGLTAGSVKDLSENTASINLNEVAQNLRYVIEQHIVAAKSTSQLANDINNFITTIPELSASSELPDESGSGQVIFVNNDNTSYADSKYRLMNRTVNNQTGNDNSDYCPEFVVGNDIDNSNPVVQAENLNWEYFLLNYGKLMGYNQDGNFDGFRIDAADDMDADVLDQIGQLMNDMYHMKGNPQNANNHLSYNEGYGPGAARMLNKKGNPQLFMDARECNTLENVLGRANNRDTISHLVTDSIVNRQNDVTENEATPNWSYVTNHDIRNNLINGLIIKDHPGMGSAYKAEYANQAWQEFYADQKKTDKQYAQYNVPAQYAILLSNKDTVPQIYYGDLYNETAQYMQEKSIYYDAITTLMKARKQFVSGGQTMTKLSDNLIASVRYGKGVANANSEGTDSLSRTSGMAVIVGNNPQMAEQTISINMGRAHANEQYRNLLDTTDNGLTYNADGAENPETLTTDDNGILKVTVKGYSNPYVSGYLGVWVPVASGNQDVTTNAATVSADSNKIFESNAALDSHMIYEDFSMYQPKPTSTENHAYNIIAQNAELFNNLGITDFWMAPAYTQAGTSRYNEGYSVADRYNLGTNANPTKYGSGEELANAIAALHSAGLKVQEDIVMNQMIGLPGQEAVTVTRADNRGMQTYVNGKTYANQMYFAYTTGGGNGQETYGGKYLSELQSKYPDLFTTRAISTGVAPDPTTHITKWSAKYENGTSLQNIGIGLAVKLANGDYAYLNDSNNKAFNTTLPETMSSTDYYANIEDN

(19) United States
(12) Patent Application Publication
(10) Pub. No.: US 2025/0019733 A1
(43) Pub. Date: Jan. 16, 2025

(54) METHOD FOR OBTAINING SOLUBLE FIBRES ENZYMATICALLY

(71) Applicant: ROQUETTE FRERES, LESTREM (FR)

(72) Inventors: Pierre LANOS, LA BASSEE (FR); Matthieu RAMETTE, ARRAS (FR); Magali REMAUD-SIMEON, RAMONVILLE-SAINT-AGNE (FR); Claire MOULIS, VIEILLEVIGNE (FR); Sandra PIZZUT-SERIN, MAS-SAINTES-PUELLES (FR); Etienne SEVERAC, PIBRAC (FR)

(21) Appl. No.: 18/684,793

(22) PCT Filed: Aug. 22, 2022
(86) PCT No.: PCT/FR2022/051595

§ 371 Date: Sep. 18, 2024

(30) Foreign Application Priority Data
Aug. 23, 2021 (FR) FR21 08822

(51) Int. Cl. C12P 19/04; A23K 20/163; A23L 33/21; C12P 19/18

(52) U.S. Cl. CPC C12P 19/04; A23K 20/163; A23L 33/21; C12P 19/18

(57) ABSTRACT

A method for preparing a mixture of poorly-digestible alpha-glucans from a substrate rich in oligosaccharides having a degree of polymerization (DP) of 4.

### TECHNICAL FIELD

The present invention relates to a method for preparing a mixture of poorly-digestible alpha-glucans from a mixture of oligosaccharides and polysaccharides.

The invention also relates to a mixture of poorly-digestible alpha-glucans.

The present invention also relates to the sequential use of a glucanotransferase capable of creating alpha (1,3) glycosidic bonds and a glucanotransferase capable of creating alpha (1,6) glycosidic bonds to reduce the digestibility of a mixture of alpha-glucans.

### PRIOR ART

Dietary fiber has an important role in human nutrition. Among dietary fibers, a distinction is made between soluble fibers, which are soluble in water and have a gelling capacity, and insoluble fibers. Soluble fibers, including branched maltodextrins, are particularly advantageous because they are poorly digestible. Because of this, their incorporation into the diet makes it possible to reduce the glycemic index of a food and to prolong the sensation of satiety. They are also endowed with prebiotic properties on the intestinal flora; in other words, they are capable of selectively promoting the growth of certain bacteria of probiotic type or the activity of the microbiota, by providing a health benefit.

Hitherto, soluble fibers, including branched maltodextrins, were mainly obtained physicochemically.

This is the case in particular with maltodextrin sold by the Applicant company under the brand name NUTRIOSE® FM10 as water-soluble fiber.

Other soluble fibers obtained physicochemically exist, such as PROMITOR® sold by the company Tate and Lyl, FIBERSOL® or LITESSE® sold by the company Dupont Nutrition and Biosciences.

Numerous studies have demonstrated that the digestibility properties were directly linked to the percentages of the various types of glycosidic bonds within the soluble fibers.

Indeed, standard maltodextrins are rapidly digestible and are defined as purified and concentrated mixtures of glucose and glucose polymers essentially linked at alpha 1→4 (hereinafter 1→4 or alpha (1,4)) with only 4 to 5% of alpha 1→6 glycosidic bonds (hereinafter 1→6 or alpha (1,6)), of extremely varied molecular weights, completely soluble in water and of low reducing power.

By increasing the percentage of alpha 1→6 or alpha 1→3 bonds, the degree of branching of the maltodextrins is increased, which makes them more resistant to digestion.

The enzymatic approach, which uses enzymes capable of promoting the creation of “branched” type bonds, has numerous advantages, in terms of safety and environmental preservation, and also offers better specificity.

Originally, most enzymatic processes for producing soluble fibers are carried out using sucrose as a substrate for the enzyme, in order to create new bonds. For example, WO2015183714 describes an enzymatic reaction from a mixture of sucrose and alpha-glucan type substrate.

At present, most enzymatic processes use amylomaltases to produce soluble fibers from starch.

It is desirable to obtain enzymatically soluble fibers from the substrate, in the absence of sucrose.

### DETAILED DESCRIPTION OF THE INVENTION

The Applicant company has then found that it is possible, from a mixture of oligosaccharides and polysaccharides, to obtain fibers of interest in human and animal nutrition, enzymatically. The Applicant company has thus developed a method which uses two particular enzymes, one capable of creating alpha (1,3) bonds and the other capable of creating alpha (1,6) bonds, sequentially, and vice versa.

In a first aspect, the present invention relates to a method for preparing a mixture of alpha-glucans comprising the following steps:
providing a substrate, said substrate being a mixture of oligosaccharides and polysaccharides having a polydispersity index of between 5 and 10, preferably between 6 and 9.5, even more preferably between 7 and 9, between 8 and 8.5, most preferably about 8.4,
a first incubation in the presence of a first enzyme,
a second incubation with a second enzyme, said first and second enzymes being an alpha-glucanotransferase capable of cleaving alpha (1,4) glycosidic bonds and creating alpha (1,3) glycosidic bonds and/or an alpha-glucanotransferase capable of cleaving alpha (1,4) glycosidic bonds and creating alpha (1,6) glycosidic bonds

According to the present invention, the terms “alpha-glucan”, “soluble fiber”, “food soluble fiber” are used interchangeably. They define oligosaccharides composed of at least 3 glucose units linked together by alpha-glycosidic (or alpha-glucosidic) bonds.

The classification of alpha-glucans is mainly based on the measurement of their reducing power, conventionally expressed by the notion of “dextrose equivalent” (“Dextrose Equivalent” or DE). On this particular point, the definition of maltodextrins given in the Monograph Specifications of the Food Chemical Codex specifies that the DE value for a maltodextrin must not exceed 20. Above 20, these are glucose syrups.

Preferably, the substrate used in the method according to the present invention has a DE of between 15 and 20, preferably between 17 and 20, preferably between 18 and 19, even more preferably about 18.4.

Such a DE measurement is however insufficient to accurately represent the molecular distribution of the alpha-glucans. Indeed, the acid hydrolysis of starch, which is totally random, or its enzymatic hydrolysis, which is slightly more ordered, provides mixtures of glucose and glucose polymers that the sole measurement of DE does not make it possible to define with precision, and which comprise molecules of short size, of low degree of polymerization (DP), as well as molecules of very long size, of high DP.

Measurement of the DE in fact gives only an approximate idea of the average DP of the mixture of glucose and of the constituent glucose polymers of the alpha-glucans and therefore of their number-average molecular mass (Mn). To complete the characterization of the molecular mass distribution of alpha-glucans, it is important to determine another parameter, that of the weight-average molecular mass (Mw).

In practice, the values of Mn and Mw are not calculated, but are measured by different techniques. For example, a measurement method suitable for glucose polymers is used, which is based on gel permeation chromatography on chromatography columns calibrated with pullulans of known molecular masses.

The Mw/Mn ratio is called the polymolecularity index or polydispersity index (PI) and makes it possible to characterize overall the molecular mass distribution of a polymer blend. As a general rule, the molecular mass distribution of standard maltodextrins results in IP values of between 5 and 10.

These various parameters are also the reflection of the alpha-glycosidic bond profile of the alpha-glucans. Indeed, a mixture of standard alpha-glucans has a very high percentage of “linear” alpha (1,4) bonds (greater than 90%) and a low percentage of so-called “branched” (alpha (1,2), alpha (1,3) and alpha (1,6) bonds).

The method according to the present invention makes it possible to reduce the percentage of alpha (1,4) bonds in favor of alpha (1,3) and alpha (1,6) bonds, which has the advantage of reducing the digestibility of the mixture of alpha-glucans obtained by the method.

According to one embodiment of the invention, the substrate comprises:
between 40 and 50% of oligosaccharides having a degree of polymerization (DP) between 1 and 9,
between 15 and 20% of polysaccharides having a DP between 10 and 20,
between 35 and 40% of polysaccharides having a DP greater than 20, the percentages being expressed as relative percentages by moles, and the total making up 100%.

Preferably, the substrate comprises:
between 90 and 97%, preferably between 92 and 95%, of alpha (1,4) bonds,
between 3 and 7%, preferably between 4 and 6%, of alpha (1,6) bonds, between 0 and 3%, preferably between 1 and 2%, of alpha (1,3) bonds, the percentage of alpha (1,6) bonds being the molar percentage of alpha (1,6) bonds respectively relative to the total number of glycosidic bonds, measured by the Hakomori method.

According to a preferred embodiment of the invention, the substrate has a dextrose equivalent (DE) of between 17 and 20, preferably between 18 and 19, even more preferably about 18.4.

According to a preferred embodiment of the invention, the substrate has the characteristics described in Table 1 below. It may be, for example, Glucidex 19D® sold by the Applicant company.

In a preferred embodiment of the invention, the substrate is present at a concentration of between 50 g/L and 500 g/L, preferably between 100 g/L and 200 g/L, in the reaction medium.

The two enzymes are used sequentially.

In one embodiment, the first enzyme is an alpha-glucanotransferase capable of cleaving alpha (1,4) glycosidic bonds, but also to cleave the alpha (1,4) glycosidic bonds and create alpha (1,3) glycosidic bonds and the second enzyme is an alpha-glucanotransferase capable of cleaving the alpha (1,4) glycosidic bonds and creating alpha (1,6) glycosidic bonds.

In another embodiment, the first enzyme is an alpha-glucanotransferase capable of cleaving alpha (1,4) glycosidic bonds and creating alpha (1,6) glycosidic bonds and the second enzyme is an alpha-glucanotransferase capable of cleaving alpha (1,4) glycosidic bonds and creating alpha (1,3) glycosidic bonds.

In a preferred embodiment of the invention, the alpha-glucanotransferase capable of hydrolyzing the alpha (1,4) glycosidic bonds and of creating alpha (1,6) glycosidic bonds is the protein having the sequence SEQ ID No: 1 or a protein having at least 90% identity with the protein having the sequence SEQ ID No: 1. Preferably, it is a protein having at least 91%, even more preferably at least 92%, at least 93%, at least 94%, at least 95%, at least 96%, at least 97%, at least 98%, at least 99%, at least 99.5%, at least 99.6%, at least 99.7%, at least 99.8%, at least 99.9% identity with the protein having the sequence SEQ ID No: 1. The sequence SEQ ID No: 1 corresponds to the Genbank accession number WP_053069107.1.

In a preferred embodiment of the invention, the alpha-glucanotransferase capable of cleaving the alpha (1,4) glycosidic bonds and of creating alpha (1,3) glycosidic bonds is the protein having the sequence SEQ ID No: 2 or a protein having at least 90% identity with the protein having the sequence SEQ ID No: 2. Preferably, it is a protein having at least 91%, even more preferably at least 92%, at least 93%, at least 94%, at least 95%, at least 96%, at least 97%, at least 98%, at least 99%, at least 99.5%, at least 99.6%, at least 99.7%, at least 99.8%, at least 99.9% identity with the protein having the sequence SEQ ID No: 2. The sequence SEQ ID No: 2 corresponds to the Genbank accession number AOR73699.1.

According to one embodiment of the invention, each enzyme is added at a concentration of between 0.01 and 1 mg/ml of reaction medium, preferably between 0.05 and 0.5 mg/mL, even more preferably about 0.1 mg/ml of reaction medium, during sequential incubations.

According to one embodiment of the invention, the substrate and each enzyme are brought into contact for a period of between 12 and 48 hours, preferably approximately 24 hours

According to one embodiment of the invention, the substrate and each enzyme are brought together at a temperature of between 2° and 40° C., preferably approximately 37° C.

According to one embodiment of the invention, the substrate and each enzyme are brought together at a pH of between 5 and 6.5, preferably between 5.5 and 6 and even more preferably approximately 5.75.

According to one aspect, the present invention also relates to a mixture of alpha-glucans which can be obtained by the method described above.

This mixture of alpha-glucans is characterized by its low digestibility according to the AOAC 2002.02 method. Advantageously, the process according to the invention makes it possible to reduce the hydrolyzable fraction, measured according to the AOAC 2002.02 method, by a factor of at least 2, preferably of at least 2.5, even more preferably of at least 3, relative to the starting substrate.

The AOAC 2002.02 method can in particular be implemented using the “HPAEC-PAD assay” part of the “Starch resistant, K-RSTAR 06/18” kit sold by the company Megazyme® as described in Example 1, part 6 below.

The method according to the present invention makes it possible to increase the percentage of alpha (1,6) bonds by a factor of at least 3, preferably at least 4, even more preferably at least 5, 6, 7 or 8, relative to the starting substrate.

The method according to the present invention also makes it possible to create alpha (1,3) bonds which were absent in the starting substrate.

The percentage of alpha (1,4), alpha (1,6), alpha (1,2) and alpha (1,3) bonds is measured by the Hakomori method (1964 HAKOMORI A Rapid Permethylation of Glycolipid, and Polysaccharide Catalyzed by Methylsulfinyl Carbanion in Dimethyl Sulfoxide) as described in example 1, part 9 below.

According to one aspect, the present invention relates to a mixture of alpha-glucans characterized in that it exhibits:
a content of hydrolyzable fibers, of less than 45%,
and/or at least 20% of alpha (1,6) bonds,
and/or at least 3% of alpha (1,3) bonds,
wherein the fiber content corresponds to the hydrolyzable (i.e. non-resistant) fraction according to the AOAC 2002.02 method and the percentage of alpha (1,6) and alpha (1,3) bonds represents the molar percentage of alpha (1,6) and alpha (1,3) bonds respectively relative to the total number of glycosidic bonds, measured by the Hakomori method.

Preferably, the content of hydrolyzable fibers is less than 44%, preferably less than 43% by weight relative to the total weight of dry matter, even more preferably less than 42%, 41%, 40%, 39%, 38%, 37%, 36%, 35%, 34%, 33%, 32%, 31%, 30%.

Preferably, the percentage of alpha (1,6) bonds, is at least 21%, preferably at least 22%, even more preferably at least 23%, at least 24%, at least 25%, at least 26%, at least 27%, at least 28%, at least 29%, at least 30%, at least 31%, at least 32%, at least 33%, at least 34%, at least 35%, the percentage of alpha (1,6) bonds being the molar percentage of alpha (1,6) bonds respectively relative to the total number of glycosidic bonds, measured by the Hakomori method.

Preferably, the percentage of alpha (1,3) bonds is at least 4%, preferably at least 4%, at least 5%, at least 6%, at least 7% and at least 8%, the percentage of alpha (1,3) bonds being the molar percentage of alpha (1,3) bonds respectively relative to the total number of glycosidic bonds, measured by the Hakomori method.

The present invention also relates to the use of a mixture of alpha-glucans obtained according to the method described above and of a mixture of alpha-glucans having the properties described above for the preparation of foods for human or animal nutrition.

Typically, the mixture of alpha-glucans of the invention can be used to promote intestinal health, blood glucose management, satiety and weight management, and sustained energy release.

Finally, in another aspect, the present invention relates to the sequential use of a glucanotransferase capable of cleaving alpha (1,4) glycosidic bonds and of creating alpha (1,6) glycosidic bonds and of a glucanotransferase capable of cleaving alpha (1,4) glycosidic bonds and of creating alpha (1,3) glycosidic bonds for decreasing the digestibility of a mixture of alpha-glucans. Preferably, said glucanotransferase has the sequence SEQ ID No: 1 or at least 90% identity with the protein having the sequence SEQ ID No: 1. Preferably, said glucanotransferase capable of cleaving the alpha (1,4) glycosidic bonds and of creating alpha (1,6) glycosidic bonds has the sequence SEQ ID No: 1 or at least 90% identity with the protein having the sequence SEQ ID No: 1. Preferably, said glucanotransferase capable of cleaving the alpha (1,4) glycosidic bonds and of creating alpha (1,3) glycosidic bonds has the sequence SEQ ID No: 2 or at least 90% identity with the protein having the sequence SEQ ID No: 2.

Preferably, the decrease in digestibility is a decrease by a factor of at least 2, preferably of at least 2.5, even more preferably of at least 3 of the hydrolyzable fraction, measured according to the AOAC 2002.02 method.

The invention will be better understood with the aid of the following examples, which are intended to be illustrative and non-limiting.

### Example 1: Preparation of Branched Maltodextrins from a Mixture of Oligosaccharides and Polysaccharides: Material and Methods

### 1. Preparation of a Substrate Solution Comprising a Mixture of Oligosaccharides and Polysaccharides

The starting substrate used was a mixture of oligosaccharides and polysaccharides, having the characteristics described in Table 1:

TABLE 1

| | | Measurement | | |
|---|---|---|---|---|
| Criteria | Sub-criteria | method | Unit | Value |
| Molecular | Mn | MCL1439 | Da | 1130 |
| weight | (pullulan eq) | | | |
| Molecular | Mw | MCL1439 | Da | 9510 |
| weight | (pullulan eq) | | | |
| Molecular | Polydispersity | MCL1439 | | 8.4 |
| weight | index | | | |
| Carbohydrate | DP1 | MCL190A | Relative % | 1.7 |
| distribution | | | | |
| Carbohydrate | DP2 | MCL190A | Relative % | 5 |
| distribution | | | | |
| Carbohydrate | DP3 | MCL190A | Relative % | 7.2 |
| distribution | | | | |
| Carbohydrate | DP4 | MCL190E | Relative % | 5.6 |
| distribution | | | | |
| Carbohydrate | DP5 | MCL190E | Relative % | 6 |
| distribution | | | | |
| Carbohydrate | DP6 | MCL190E | Relative % | 7 |
| distribution | | | | |
| Carbohydrate | DP7 | MCL190E | Relative % | 6.1 |
| distribution | | | | |
| Carbohydrate | DP8 | MCL190E | Relative % | 4.3 |
| distribution | | | | |
| Carbohydrate | DP9 | MCL190E | Relative % | 3.1 |
| distribution | | | | |
| Carbohydrate | DP10 to | MCL190E | Relative % | 17.1 |
| distribution | DP20 | | | |
| Carbohydrate | >DP20 | MCL190E | Relative % | 37 |
| distribution | | | | |
| Reducing | Dextrose | MCL050B | | 18.4 |
| sugars | equivalent | | | |
| | (DE) | | | |
| Dry material | Loss of mass | MCL209A | % | 4% |
| | after drying | | | |

Various substrate solutions in 50 mM sodium acetate buffer, pH 5.75, were prepared at concentrations of 100 g/L, 200 g/L or 400 g/L.

### 2. Production of Recombinant Enzymes.

The following enzymes were recombinantly produced;
Enzyme GT #11: alpha-4,3 glucanotransferase from Lactobacillus fermentum NC2970, of the glycoside hydrolase family GH70 having as amino acid sequence the sequence listed in Genbank under the reference AOR73699.1.
Enzyme GT #19: alpha-4,6 glucanotransferase from Lactobacillus mucosae, of the family of glycoside hydrolases GH70 having as amino acid sequence the sequence listed in Genbank under the reference WP-053069107.1.

E. coli BL21 star (DE3) cells containing plasmid pET-21 a-enzyme (in order to produce various enzymes, including GT #11 and GT #19) were cultured in ZYM-50524 medium containing 1% glycerol and 1% lactose. At the end of the culture, the cells were centrifuged at 6500 g for 10 minutes, the cell pellets resuspended at a DO600 nm of 80 in a 20 mM phosphate buffer, pH 7.4, containing 300 mM of NaCl and 20 mM of imidazole, and the cells were lysed by cold sonication using 4 cycles of 20 seconds at 30% amplitude followed by 4 minutes of rest. The cell debris was separated from the solubilized proteins by centrifugation for 30 minutes at 10,000 g.

### 3 Purifying Enzymes

Purification of the proteins of interest was carried out on Cobalt resin (Invitrogen) loaded with divalent cobalt ions (CO²⁺), for which the polyhistidine tag has an affinity. Elution was carried out by creating a competition between the polyhistidine tag and increasing concentrations of imidazole. Briefly, 10 to 35 mL of cell extract of E. coli were brought into contact for 1 hour with 1 mL of Cobalt resin equilibrated beforehand with 25 mL of 20 mM Phosphate buffer, pH 7.4, containing 300 mM of NaCl and 20 mM of imidazole. Filtration of the resin on sintered glass allows all of the unbound proteins to be removed. The resin was then washed 5 times with 40 mL of 20 mM Phosphate buffer, pH 7.4, containing 300 mM NaCl and 20 mM imidazole. Finally, elution was carried out with 3 mL of 20 mM Phosphate buffer, pH 7.4, containing 300 mM NaCl and 250 mM imidazole for 5 minutes in order to detach the enzymes of interest. The enzymatic solutions were then dialyzed (sigma 10 kDa membrane) against 5 L of 50 mM sodium acetate buffer, pH 5.75, containing 150 Mm of NaCl (overnight, 4° C. with stirring) in order to remove the NaCl and imidazole. The various protein solutions were assayed by measuring their absorbance at 280 nm using a 2000 spectrophotometer nanodrop (Thermofisher). The molecular extinction coefficients & were determined using the ProtParam tool application of the ExPASy bioinformatics resource portal site.

Electrophoresis under denaturing conditions made it possible to control the quality of the purified enzyme extracts. For this purpose, samples containing 30 μl of protein extract and 10 μl of the loading buffer (NuPage LDS Sample buffer 4×, Invitrogen) were denatured for 5 minutes at 95° C. and then deposited on precast acrylamide gels (Mini-Protean Tris-Glycine extented (Biorad)). Migration was carried out for 30 minutes in 1×Tris/Glycine/SDS buffer under a voltage of 150 V. The proteins were then revealed by incubating the gels for 1 hour in a staining solution (PageBlue Protein Staining Solution, Fermentas) and then by rinsing for 30 minutes in three consecutive baths of water.

### 4. Branching Enzyme Activity Measurements

The enzymatic activity of the branching enzymes can be determined by measuring the initial rate of production of the reducing sugars using the dinitrosalicylic acid (DNS) method. One enzyme unit represents the amount of enzyme which releases one pmol of fructose per minute, at 30° C. for an initial sucrose concentration of 100 g·L-1 under the buffer conditions of adequate activity. During a kinetics of 1 mL of volume, 100 μL of reaction medium were removed and the reaction stopped by adding an equivalent volume of DNS. The samples were then heated for 5 minutes at 95° C., cooled in ice, diluted to half in water, and absorbance was read at 540 nm. A standard range of 0 to 2 g·L-1 of fructose makes it possible to establish the relationship between the absorbance value and the concentration of reducing sugars.

### 5. Enzymatic Reactions

The reactions were carried out with 0.1 mg/mL of purified enzyme either GT #11 or GT #19 and dialyzed in the presence of 10%, 20% or 40% of substrate in 50 mM sodium acetate buffer, pH 5.75. The reactions were incubated with stirring for 24 hours at 20° C. or 37° C. The reactions were stopped by heating (95° C. for 5 minutes). Samples were taken at the initial and final times to analyze the specificity of the enzymes using different analytical techniques (HPAEC-PAD, NMR and HPSEC).

### 6. Digestibility Test

The transfer reactions were freeze-dried after freezing at −80° C. for 24 hours. 25 mg of freeze-dried products were taken up in 1 mL of 100 mM sodium maleate buffer containing 30 U of pancreatic alpha-amylase and 3 U of amyloglucosidase (Starch resistant kit, Megazyme K-STAR 06/18, which implements the AOAC 2002.02 method). The reactions were incubated for 16 hours at 37° C. The products were diluted in water before HPAEC PAD analysis.

### 7. Chromatographic Analyses

The products obtained were analyzed by anion exchange chromatography coupled to a pulsed amperometric detector (HPAEC PAD-HIGH Performance Anion Exchange Chromatography with Pulsed Amperometric Detection). The analyses were carried out on a Thermo ICS6000 system equipped with a CarboPac™ PA100 analytical column (2 mm×250 mm) coupled with a CarboPac™ PA100 guard pre-column (2 mm×50 mm). A gradient of sodium acetate in 150 mM sodium was applied at a flow rate of 0.250 ml·min-1 according to the following profile: 0-5 min, 0 mM; 5-35 min, 0-300 mM; 35-40 min, 300-450 mM; 40-42 min, 450 mM. Detection was carried out using a gold working electrode and a pH Ag/AgCl reference cell. The samples were diluted to a total dry mass of 1 g·L-1 before injection. The size of the reaction products was also sometimes determined by High Performance Size Exclusion Chromatography on a Fisher Ultimate 3000 system equipped with a Shodex OH-Pak SB-802.5 column protected by a Shodex OH-Pak SB-G guard column pre-column, placed at 70° C. in the oven of the system. The mobile phase was water at a flow rate of 0.3 mL·min-1. The detection was carried out by refractometry. The samples were diluted to a total dry mass of 20 g·L-1 before injection.

### 8. NMR.

Spectra ¹H, ¹³C and HSQC were recorded on a Bruker Avance 500 MHz equipment at 298 K with a 5 mm Z-gradient H-BB-D BBI probe. The data were acquired and processed using the TopSpin 3 software.

### 9. Hakomori Method

The Hakomori method (1964 HAKOMORI A Rapid Permethylation of Glycolipid, and Polysaccharide Catalyzed by Methylsulfinyl Carbanion in Dimethyl Sulfoxide) makes it possible to chemically characterize the glycosidic bonds by differentiating the free OH groups and the bonded groups. This is a destructive method comprising the steps of methylation, hydrolysis, reduction with NaBD4, acetylation and analysis by mass spectrometry.

### Example 2: Separate Use of GT #11 and GT #19 Enzymes

In this example, the action of the GT #11 and GT #19 enzymes was tested separately.

The results of the various enzymatic reactions are presented in Table 2 below, which shows the percentages of alpha-1,6; alpha-1,3 and alpha-1,4 bonds measured by proton NMR or by the Hakomori method and percentage of hydrolysis (AOAC 2002.02) in the reaction products obtained.

TABLE 2

| | | | | | | | | | | % hydrolysis |
|---|---|---|---|---|---|---|---|---|---|---|
| | Concentration | NMR | | | | HAKOMORI | | | | (AOAC |
| Enzyme | g/L | α-1,2 | α-1,3 | α-1,4 | α-1,6 | α-1,2 | α-1,3 | α-1,4 | α-1,6 | 2002.02) |
| / | 100 | 0% | 0% | 95% | 5% | | | | | 88% |
| / | 200 | 0% | 0% | 95% | 5% | | | | | 94% |
| / | 400 | 0% | 0% | 95% | 5% | | | | | 86% |
| GT#11 | 100 | 0% | 17% | 72% | 11% | 12% | 8% | 78% | 12% | 58% |
| GT#11 | 200 | 0% | 15% | 76% | 9% | 3% | 8% | 77% | 12% | 68% |
| GT#11 | 400 | 0% | 14% | 78% | 8% | 2% | 5% | 84% | 10% | 96% |
| GT#19 | 100 | 0% | 0% | 65% | 35% | 1% | 3% | 66% | 30% | 46% |
| GT#19 | 200 | 0% | 0% | 69% | 31% | 6% | 6% | 62% | 26% | 55% |
| GT#19 | 400 | 0% | 0% | 73% | 27% | 2% | 3% | 75% | 20% | 63% |

The inventors observed that the enzyme GT #11 was capable of reducing the percentage of alpha-1,4 linear bonds and increasing the percentage of so-called “branched” alpha-1,3 and alpha-1,6 bonds.

Enzyme GT #19 was, for its part, capable of decreasing the percentage of linear alpha-1,4 bonds and of markedly increasing the percentage of so-called “branched” alpha-1,6 bonds.

In both cases, an increase in the resistance to digestion (reflected by a decrease in the degree of hydrolysis) was observed. However, the products obtained are not strong enough to be considered as fibers.

### Example 3: Simultaneous Use of GT #11 and GT #19 Enzymes

In this example, the inventors studied the combined action of the two enzymes GT #11 and GT #19.

The two enzymes were therefore added simultaneously to the reaction mixture, in the various proportions mentioned in the left-hand column of Table 3.

The results of the various enzymatic reactions are presented in Table 3 below, which shows the percentages of alpha-1,6; alpha-1,3 and alpha-1,4 bonds measured by proton NMR or by the Hakomori method and percentage of hydrolysis (AOAC 2002.02) in the reaction products obtained.

TABLE 3

| | | | | | | | | | | % hydrolysis |
|---|---|---|---|---|---|---|---|---|---|---|
| | Concentration | NMR | | | | HAKOMORI | | | | (AOAC |
| Enzyme | g/L | α-1,2 | α-1,3 | α-1,4 | α-1,6 | α-1,2 | α-1,3 | α-1,4 | α-1,6 | 2002.02) |
| 0.05 mg/mL | 200 | 0% | 0% | 64% | 36% | 2% | 5% | 65% | 28% | 55% |
| GT#11 + | | | | | | | | | | |
| 0.05 mg/mL | | | | | | | | | | |
| GT#19 | | | | | | | | | | |
| 0.075 mg/mL | 200 | 0% | 7% | 65% | 28% | 2% | 4% | 67% | 27% | 52% |
| GT#11 + | | | | | | | | | | |
| 0.025 mg/mL | | | | | | | | | | |
| GT#19 | | | | | | | | | | |
| 0.025 mg/mL | 200 | 0% | 0% | 66% | 34% | 1% | 4% | 64% | 31% | 57% |
| GT#11 + | | | | | | | | | | |
| 0.075 mg/ml | | | | | | | | | | |
| GT#19 | | | | | | | | | | |

The inventors observed that the simultaneous action of the enzymes GT #1 and GT #19 resulted in a decrease in the percentage of linear alpha-1,4 bonds and an increase in the percentage of so-called “branched” alpha-1,3 and alpha-1,6 bonds. The results obtained are equivalent, or even slightly less good than the use of GT19 alone on 200 g/L of substrate.

This modification of the binding profile results in an increase in the resistance to digestion (reflected by a decrease in the degree of hydrolysis) was observed. However, the products obtained are not sufficiently resistant (<40% hydrolysis according to the AGAC2002.02 method) to be considered as fibers.

### Example 4: Sequential Use of GT #11 and GT #19 Enzymes

In this example, the inventors studied the sequential action of the two enzymes GT #11 and GT #19.

The enzyme cascade represents a good strategy for increasing the resistance of the products to hydrolytic enzymes and achieving a digestibility level of less than 40%.

Within the framework of the enzyme cascade, the enzymes are used one after the other. Two different configurations alternating the two alpha-GT were studied:
Glucidex 19 D is dissolved at 200 g·L-1, the first alpha-GT is reacted at a concentration of 0.05 mg·mL-1 for 24 hours. The reaction is stopped by heating for 5 minutes at 95° C. The second enzyme is then reacted at the same concentration of 0.05 mg·mL-1. The reaction is again stopped by heating for 5 minutes at 95° C. incubation for 24 hours.
Glucidex 19D is reacted at 100 g·L-1, first alpha-GT is reacted at a concentration of 0.05 g·L-1 for 24 hours. The reaction is stopped by heating for 5 minutes at 95° C. The reaction medium is supplemented with 100 g·L-1 of Glucidex 19D and the second enzyme is then reacted at the same concentration of 0.05 g·L-1. The reaction is again stopped by heating for 5 minutes at 95° C. incubation for 24 hours.

These various strategies make it possible to take advantage of the 4,3-alpha-glucanotransferase specificity of alpha-GT No. 11 and promote its action relative to that of alpha-GT No. 19. In fact, a non-negligible level of alpha-1,3 bonds can be achieved in addition to the level of alpha-1,6 bonds (Table 4).

The inventors have observed that levels of alpha-1,4 bonds of 50% or less are obtained under these conditions and that the three types of glycosidic bonds (alpha-1,6; alpha-1,3 and alpha-1,4) are represented in the final product

The results of the various enzymatic reactions are presented in Table 4 below, which shows the percentages of alpha-1,6; alpha-1,3 and alpha-1,4 bonds measured by proton NMR or by the Hakomori method and percentage of hydrolysis (AOAC 2002.02) in the reaction products obtained.

TABLE 4

| | | | | | | | | | | % hydrolysis |
|---|---|---|---|---|---|---|---|---|---|---|
| | Concentration | NMR | | | | HAKOMORI | | | | (AOAC |
| Enzyme | g/L | α-1,2 | α-1,3 | α-1,4 | α-1,6 | α-1,2 | α-1,3 | α-1,4 | α-1,6 | 2002.02) |
| GT#11 | 100 + 100 | 0% | 16% | 50% | 35% | 2% | 7% | 62% | 29% | 39% |
| then | | | | | | | | | | |
| GT#19 | | | | | | | | | | |
| GT#19 | 100 + 100 | 0% | 13% | 44% | 43% | 3% | 5% | 55% | 36% | 39% |
| then | | | | | | | | | | |
| GT#11 | | | | | | | | | | |
| GT#11 | 200 | 0% | 18% | 51% | 32% | 2% | 8% | 64% | 26% | 36% |
| then | | | | | | | | | | |
| GT#19 | | | | | | | | | | |
| GT#19 | 200 | 0% | 14% | 42% | 44% | 2% | 5% | 57% | 36% | 37% |
| then | | | | | | | | | | |
| GT#11 | | | | | | | | | | |

Thus, the inventors have demonstrated that the sequential use of the two enzymes, regardless of the order of this sequence, and with or without addition of substrate between the two reactions, made it possible to obtain products having a hydrolysis of less than 40%. In other words, the sequential use of alpha-glucanotransferase GT #11 and GT #19 made it possible to obtain soluble fibers from a mixture of oligosaccharides and polysaccharides having a DE of 19.

### Example 5: Sequential Use of GT #11 and GT #19 Enzymes on a Large Scale

In this example, the inventors carried out an increase in scale in order to produce 1 g of fibers instead of the 50 mg produced in the preceding examples): 15 ml of Glucidex 19D at 200 g·L-1 underwent a cascade reaction involving first alpha-GT No. 11 for 24 hours followed by alpha-GT No. 19 for 24 hours. Each enzyme was used at 0.1 g·L-1. The same distribution in type of bonds is obtained relative to the smaller volume equivalent test of Example 4 (Table 5):

TABLE 5

| | Concentration | NMR | | | | HAKOMORI | | | |
|---|---|---|---|---|---|---|---|---|---|
| Enzyme | g/L | α-1,2 | α-1,3 | α-1,4 | α-1,6 | α-1,2 | α-1,3 | α-1,4 | α-1,6 |
| GT#11 then | 200 | 0% | 18% | 56% | 26% | 2% | 8% | 68% | 22% |
| GT#19 | | | | | | | | | |

1. A method for preparing a mixture of a-glucans comprising the following steps:
providing a substrate, said substrate being a mixture of oligosaccharides and polysaccharides having a polydispersity index of between 5 and 10, preferably between 6 and 9.5, even more preferably between 7 and 9, between 8 and 8.5, most preferably about 8.4,
a first incubation in the presence of a first enzyme,
a second incubation with a second enzyme,
said first and second enzymes being an α-glucanotransferase capable of cleaving alpha (1,4) glycosidic bonds and creating α(1,3) glycosidic bonds and/or an α-glucanotransferase capable of cleaving α(1,4) glycosidic bonds and creating α(1,6) glycosidic bonds.

2. The method according to claim 1, wherein the substrate comprises:
between 40 and 50% of oligosaccharides having a degree of polymerization (DP) between 1 and 9,
between 15 and 20% of polysaccharides having a DP between 10 and 20,
between 35 and 40% of polysaccharides having a DP greater than 20,
the percentages being expressed as relative percentages by moles, and the total making up 100%

3. The method according to claim 1 or 2, wherein the substrate has a dextrose equivalent (DE) between 18 and 20, preferably between 18 and 19, even more preferably approximately 18.4.

4. The method according to any one of the preceding claims, wherein the substrate is introduced at a concentration of between 50 g/L and 500 g/L, preferably between 100 g/L and 200 g/L of reaction medium.

5. The method according to any one of the preceding claims, wherein the substrate is added between the first and second incubations.

6. The method according to any one of the preceding claims, wherein the first enzyme is an α-glucanotransferase capable of cleaving the α(1,4) glycosidic bonds and creating α(1,3) glycosidic bonds and the second enzyme is an α-glucanotransferase capable of cleaving the α(1,4) glycosidic bonds and creating α(1,6) glycosidic bonds.

7. The method according to any one of claims 1 to 5, wherein the first enzyme is an α-glucanotransferase capable of cleaving the α(1,4) glycosidic bonds and creating α(1,6) glycosidic bonds and the second enzyme is an α-glucanotransferase capable of cleaving the α(1,4) glycosidic bonds and creating α(1,3) glycosidic bonds.

8. The method according to any one of the preceding claims, wherein the α-glucanotransferase capable of cleaving the α(1,4) glycosidic bonds and creating α(1,6) glycosidic bonds is the protein having the sequence SEQ ID No: 1 or a protein having at least 90% identity with the protein having SEQ ID No: 1.

9. The method according to any one of the preceding claims, wherein the α-glucanotransferase capable of cleaving the α(1,4) glycosidic bonds and of creating α(1,3) glycosidic bonds is the protein having the sequence SEQ ID No: 2 or a protein having at least 90% identity with the protein having the sequence SEQ ID No: 2.

10. The method according to any one of the preceding claims, wherein each enzyme is at a concentration of between 0.01 and 1 mg/ml of reaction medium, preferably between 0.05 and 0.5 mg/mL, even more preferably approximately 0.1 mg/ml of reaction medium.

11. The method according to any one of the preceding claims, characterized in that each incubation is carried out for a period of between 12 and 48 hours, preferably approximately 24 hours and/or at a temperature comprised between 2° and 40° C., preferably approximately 37° C. and/or at a pH of between 5 and 6.5, preferably approximately 5.75.

12. A mixture of α-glucans capable of being obtained by the method according to any one of the preceding claims.

13. A mixture of α-glucans characterized in that it has:
a content of hydrolyzable fibers less than 45%, by weight relative to the total weight of dry matter,
and/or at least 20% of a (1,6) bonds,
and/or at least 3% of a (1,3) bonds,
wherein the fiber content corresponds to the hydrolyzable (i.e. non-resistant) fraction according to the AOAC 2002.02 method and the percentage of α(1,6) and α(1,3) bonds represents the molar percentage of α(1,6) and α(1,3) bonds respectively relative to the total number of glycosidic bonds, measured by the Hakomori method.

14. Use of a mixture of α-glucans according to any one of claim 12 or 13 for preparing food for human or animal nutrition.

15. Sequential use of a glucanotransferase capable of cleaving the α(1,4) glycosidic bonds and of creating α(1,6) glycosidic bonds and of a glucanotransferase capable of cleaving the α(1,4) glycosidic bonds and of creating α(1,3) glycosidic bonds to reduce the digestibility of a mixture of α-glucans, said glucanotransferases respectively having the sequence SEQ ID No: 1 or a protein having at least 90% identity with the protein having SEQ ID No: 1 and SEQ ID No: 2 or a protein having at least 90% identity with the protein having SEQ ID No: 2.
